(19) United States
(12) Patent Application Publication
(10) Pub. No.: US 2014/0236085 A1
(43) Pub. Date: Aug. 21, 2014

(54) DEVICE FOR A SYRINGE

(71) Applicant: Wayne LANDSBERG, (US)

(72) Inventor: Wayne Landsberg, Durban (ZA)

(73) Assignee: Epikwik International AG, Zug (CH)

(21) Appl. No.: 14/347,910

(22) PCT Filed: Oct. 3, 2012
(86) PCT No.: PCT/ZA2012/000061

§ 371 Date: Mar. 27, 2014

(30) Foreign Application Priority Data
Oct. 10, 2011 (ZA) 2011/07422

(51) Int. Cl. A61B 17/34; A61M 5/20

(52) U.S. Cl. CPC A61B 17/3401; A61M 5/2033
USPC 604/135

(57) ABSTRACT

The invention provides an accessory (10) for a Loss of Resistance Syringe (20), the accessory comprising a coiled spring (12) attachable to the exterior of the syringe for biasing the plunger (22) towards the barrel (24) of the syringe. When the plunger of the syringe is withdrawn, the spring is subjected to a compressive force against which it will act. In use, as the syringe enters the epidural space, a loss of resistance will allow the spring to pull the plunger back towards the barrel of the syringe, and alerting the user not to push the needle any further.

### BACKGROUND OF THE INVENTION

1. Field of the Invention

This invention relates to an accessory for a syringe.

2. Discussion of Related Art

Epidural anesthesia or analgesia involves injection of drugs through a catheter placed into the epidural space. It has become a popular means of anesthetic for a number of medical procedures including surgical and obstetric. The injection blocks the transmission of signals through nerves in or near the spinal cord to cause both a loss of sensation (anesthesia) and a loss of pain (analgesia).

The epidural space is the space inside the bony spinal canal but outside the membrane called the dura mater (sometimes called the “dura”). In contact with the inner surface of the dura is another membrane called the arachnoid mater (“arachnoid”). The arachnoid encompasses the cerebrospinal fluid that surrounds the spinal cord.

Insertion of an epidural involves threading a needle between the vertebrae and into the epidural space taking great care to avoid puncturing the dura mater and arachnoid layers immediately below the epidural space and containing cerebrospinal fluid under pressure.

The technique commonly employed in inserting an epidural needle is known as the Loss of Resistance or LOR technique.

This technique requires an anesthetist to draw an amount of air or saline into the barrel of the epidural syringe and to then carefully push the syringe needle through the layers of back tissues and ligaments while applying a constant pressure to the plunger of the syringe towards the barrel of the syringe. As the syringe enters the epidural space, a loss of resistance is felt against the plunger which is consequently depressed, causing the air or saline to be injected into the epidural space, and alerting the anesthetist not to push the needle any further. This technique works because the ligamentum flavum is extremely dense, and injection into it is almost impossible. In the epidural space, on the other hand, there is negative or neutral pressure.

The LOR technique requires great skill and is made even more difficult by the fact that the anesthetist has to maintain one thumb on the plunger and use the other hand to insert the needle.

Attempts have been made to overcome this problem by providing syringes that are positively pressurised via a one way valve, or that include an elastomeric band attached over the plunger of the syringe such that the plunger is biased for return from a drawn position to a depressed position for injecting the syringe contents, when loss of resistance is felt. Another alternative includes a spring located inside the syringe housing. However some of these applications have not proved successful, while others involve the introduction of completely new technology, requiring the replacement of existing epidural syringes with new stock.

It is an object of this invention to provide an accessory for an existing syringe which, at least partially, alleviates some of the abovementioned problems.

### SUMMARY OF THE INVENTION

In accordance with this invention, there is provided an accessory for a Loss of Resistance Syringe having a plunger slidably located in a barrel, the accessory comprising a coiled spring attachable to the exterior of the syringe for biasing the plunger towards the barrel of the syringe.

In its simplest embodiment, one end of the spring may be attachable to the barrel of the syringe, and the other end of the spring may be attachable to the plunger of the syringe such that the action of drawing the plunger out of the syringe will subject the spring to a force and cause it to exert an opposite biasing force on the plunger in the direction of the barrel.

The accessory may include a support means for supporting the spring.

The support means may include a shaft around which the spring is located, alternatively the support means may include a housing locatable around the spring.

The support means preferably includes both a shaft around which the spring is located and a housing locatable around the spring, the shaft being slidably movable in and out the housing, and connected to at least one end of the spring, movement of the shaft in or out the housing resulting in a force to be applied to the spring.

The support means may be attachable to the barrel and/or plunger of the syringe by attachment means (so that the action of drawing the plunger of the syringe out of the barrel subjects the spring to a force, causing it to exert an opposite biasing force on the plunger in the direction of the barrel).

The attachment means may include one or more clips shaped to fit around the barrel and/or plunger of the syringe. The attachment means preferably includes at least two clips, one connecting the shaft of the accessory to the plunger of the syringe, and the other connecting the housing of the accessory to the barrel of the syringe, preferably around the flange located at the entrance to the barrel.

The attachment means further preferably includes a third clip locatable along the housing, for attachment to the barrel.

In one embodiment of the invention, the coiled spring may be subjected to a compressive force when the plunger is drawn out of the barrel—causing the spring to exert a force in the opposite direction.

In an alternative embodiment of the invention, the spring may be subjected to a stretching force when the plunger is pulled away from the barrel—causing it to exert a compressive force, pulling the plunger back towards the barrel.

### BRIEF DESCRIPTION OF THE DRAWINGS

A preferred embodiment of the invention is described below by way of example only and with reference to the following drawings, in which:

FIG. 1 is a representation of the accessory prior to attachment to the syringe;

FIG. 2 is a further representation of the accessory attached to the syringe;

FIG. 3 is a representation of the accessory in use.

### DETAILED DESCRIPTION OF THE INVENTION

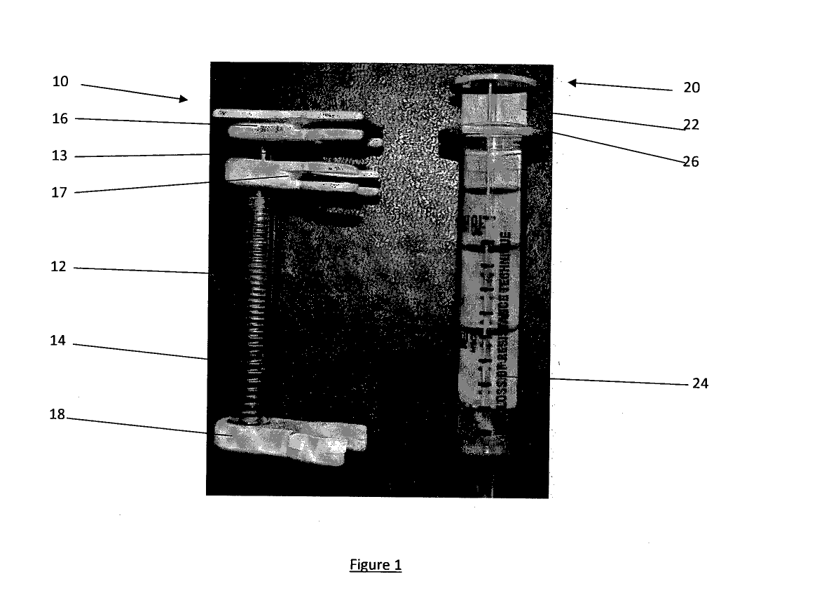

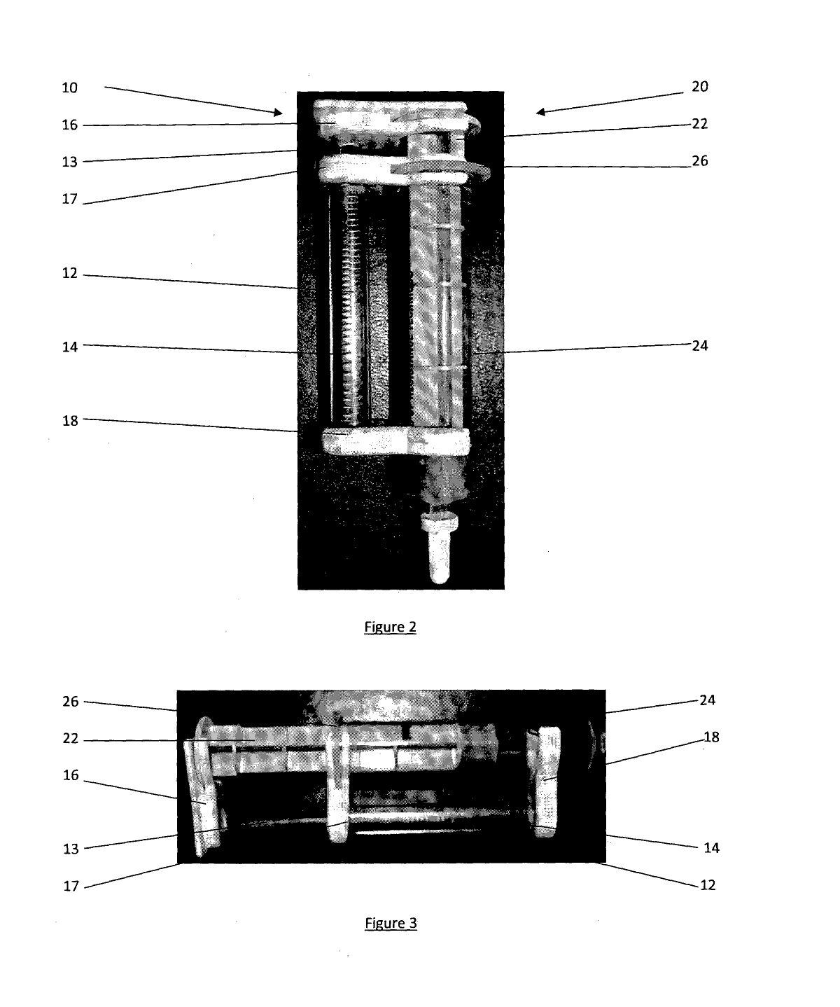

Referring to FIGS. 1, 2 and 3 in which like features of the invention are indicated by like numerals.

An accessory 10 for a Loss or Resistance Syringe 20 is disclosed. The syringe 20 has a plunger 22 slidably located in a barrel 24. The accessory 10 comprises a coiled spring 12, located on a shaft 13, enclosed in a housing 14 and attachable to the exterior of the syringe 20.

Clip 16 is located at the head of the shaft 13, and attaches the shaft 13 to the plunger 22 of the syringe 20. Clip 17 is located at the top end of the housing 14, and attaches the housing to the top of the barrel 24 of the syringe 20—either just below or around the flange 26 of the barrel 24. Clip 18 attaches the housing 14 at a further point along the barrel 24 of the syringe 20.

In use, and referring to FIG. 3, when the plunger 22 of the syringe 20 is withdrawn, the shaft 13 of the accessory 10 is withdrawn from the housing 14 of the accessory 10. This in turn causes the spring 12 to be subject to a compressive force against which it will act. The housing 14 is retained in place by the clips 17 and 18.

The anesthetist will usually draw a small amount of saline fluid (about 5 mls) into the syringe.

While the syringe needle (preferably a Tuohy needle) is being pushed through the layers of back tissues and ligaments, resistance encountered will be strong enough to maintain the spring and thus the syringe in the loaded position. However, as the syringe enters the epidural space, a loss of resistance allows the spring to pull the plunger back towards the barrel of the syringe, causing the saline to be injected into the epidural space, and at the same time alerting the anesthetist not to push the needle any further.

Numerous modifications to this embodiment are possible, without departing from the scope of the invention. For example, the base of the spring may be fixed within the housing, and the top of the spring may be pulled by the shaft, such that the spring is subjected to a stretching force when the plunger is pulled away from the barrel—causing it to exert a compressive force, pulling the plunger back towards the barrel.

The invention therefore provides a novel accessory to an existing Loss of Resistance Syringe.

1. An accessory for a Loss of Resistance Syringe having a plunger slidably located in a barrel, the accessory comprising:
a coiled spring attachable to the exterior of the syringe for biasing the plunger towards the barrel of the syringe.

2. The accessory for a Loss of Resistance Syringe as claimed in claim 1, wherein one end of the coiled spring is attachable to the barrel of the syringe, and the other end of the coiled spring is attachable to the plunger of the syringe such that the action of drawing the plunger out of the syringe will subject the spring to a force and cause it to exert an opposite biasing force on the plunger in the direction of the barrel.

3. The accessory for a Loss of Resistance Syringe as claimed in claim 1 wherein the accessory includes a means for supporting the spring.

4. The accessory for a Loss of Resistance Syringe as claimed in claim 3 wherein the support means includes a shaft around which the spring is located,

5. The accessory for a Loss of Resistance Syringe as claimed in claim 3 wherein the support means includes a housing locatable around the spring.

6. The accessory for a Loss of Resistance Syringe as claimed in claim 3 wherein the support means includes both a shaft around which the spring is located and a housing locatable around the spring, the shaft being slidably movable in and out the housing, and connected to at least one end of the spring, movement of the shaft in or out the housing resulting in a force to be applied to the spring.

7. The accessory for a Loss of Resistance Syringe as claimed in claim 3 wherein the support means is attachable to the barrel and/or plunger of the syringe by attachment means so that the action of drawing the plunger of the syringe out of the barrel subjects the spring to a force, causing it to exert an opposite biasing force on the plunger in the direction of the barrel.

8. The accessory for a Loss of Resistance Syringe as claimed in claim 7 wherein the attachment means includes one or more clips shaped to fit around the barrel and/or plunger of the syringe.

9. The accessory for a Loss of Resistance Syringe as claimed in claim 8 wherein the attachment means includes at least two clips, a first clip connecting the shaft of the accessory to the plunger of the syringe, and a second clip connecting the housing of the accessory to the barrel of the syringe.

10. The accessory for a Loss of Resistance Syringe as claimed in claim 9 wherein the second clip is located around a flange located at the entrance to the barrel of the syringe.

11. The accessory for a Loss of Resistance Syringe as claimed in claim 9 wherein the attachment means includes a third clip eatable along the housing, for attachment to the barrel.

12. The accessory for a Loss of Resistance Syringe as claimed in claim 1 wherein the coiled spring is subjected to a compressive force when the plunger is drawn out of the barrel, causing the spring to exert a force in the opposite direction.

13. The accessory for a Loss of Resistance Syringe as claimed in claim 1 wherein the coiled spring is subjected to a stretching force when the plunger is pulled away from the barrel, causing it to exert a compressive force, pulling the plunger back towards the barrel.
